(19) United States
(12) Patent Application Publication
(10) Pub. No.: US 2008/0065218 A1
(43) Pub. Date: Mar. 13, 2008

(54) ANNULUS FIBROSUS REPAIR DEVICES AND TECHNIQUES

(76) Inventor: Michael J. O'Neil, West Barnstable, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/531,302

(22) Filed: Sep. 13, 2006

(51) Int. Cl. A61F 2/44

(52) U.S. Cl. 623/17.16

(57) ABSTRACT

This invention relates to the repair and regeneration of the intervertebral annulus fibrosus damaged, for example, through surgical intervention including, discectomy and/or implantation of devices in the intervertebral disc space and a method for stabilizing a defective intervertebral nucleus implant in an intervertebral disc with a defective or damaged annulus fibrosus.

### BACKGROUND OF THE INVENTION

1. Field of the Invention

This invention is concerned with repair of intervertebral discs, particularly with repair and/or regeneration of the annulus fibrosus of an intervertebral disc.

2. Related Art

Annular wall trauma caused from discectomy or graft/device implantation punctures and weakens the annulus, reduces Functional Spinal Unit (FSU) stiffness and may result in loss of disc height. The annular damage from device insertion can also allow for expulsion of grafts through the annular wall.

U.S. Pat. No. 6,224,630 (Bao) discloses a porous annular plug produced from synthetic as well as natural biodegradable polymers including collagen for sealing the aperture and permitting natural tissue ingrowth. The plug can be attached with suture, staples, sealants or glues. The device of this invention differs from this art as it may utilize both intra-annular and extra-annular reinforcement straps for secure attachment (either separately or together) and/or an intra-annular resorbable reinforcement band for additional plug securement.

Some advantages of the device of this method include a matrix for regenerating and sealing the annulus to minimize device expulsions, to maintain disc height post disc surgery, to provide a technique for release of prophylactic agents, and/or to enable disc arthroplasty revision techniques.

### BRIEF DESCRIPTION OF THE DRAWINGS

FIG. 1 depicts a preferred embodiment of the device of this invention.

FIG. 2 depicts the insertion of the device of this invention.

FIG. 3 depicts a isometric view of the device shown in FIG. 1 as deployed.

FIG. 4 depicts a vertical view of the deployed device of FIG. 2.

FIG. 5 depicts a multi-layered regeneration matrix embodiment of this invention.

FIG. 6a depicts modular regeneration matrices of this invention.

FIG. 6b depicts modular regeneration matrices of this invention.

FIG. 7 depicts revision surgery using the device of this invention.

### SUMMARY OF THE INVENTION

A device for repair and/or regeneration of the annulus fibrousus of an intervertebral disc comprising:
a) a regeneration matrix sized and shaped to form a part of the annulus fibrosus;
b) the regeneration matrix further comprising a strap or projection to secure the material to the annulus fibrosus.

The invention is also directed to modular parts of the device of the invention which may be assembled in the operating room to fit the particular requirements of a patient.

Also, the device of this invention may be used in a method to securely close the annulus fibrosus during a total athroplasty revision.

### DETAILED DESCRIPTION OF PREFERRED EMBODIMENTS OF THE INVENTION

The device of this invention is best described as an annular plug is used to replace and regenerate the annulus. The annulus optimally requires repair and regeneration following a surgical procedure where the integrity of the annular wall has been compromised to ensure load transfer, prevent intra-annular device explusion, minimize disc height loss and allow for potential subsequent revision techniques. Preferred embodiments of this invention include an annular plug comprising intra-annular straps, a annular wall regeneration matrix and extra-annular straps as well as optional components such as fasteners and an intra-annular reinforcement band.

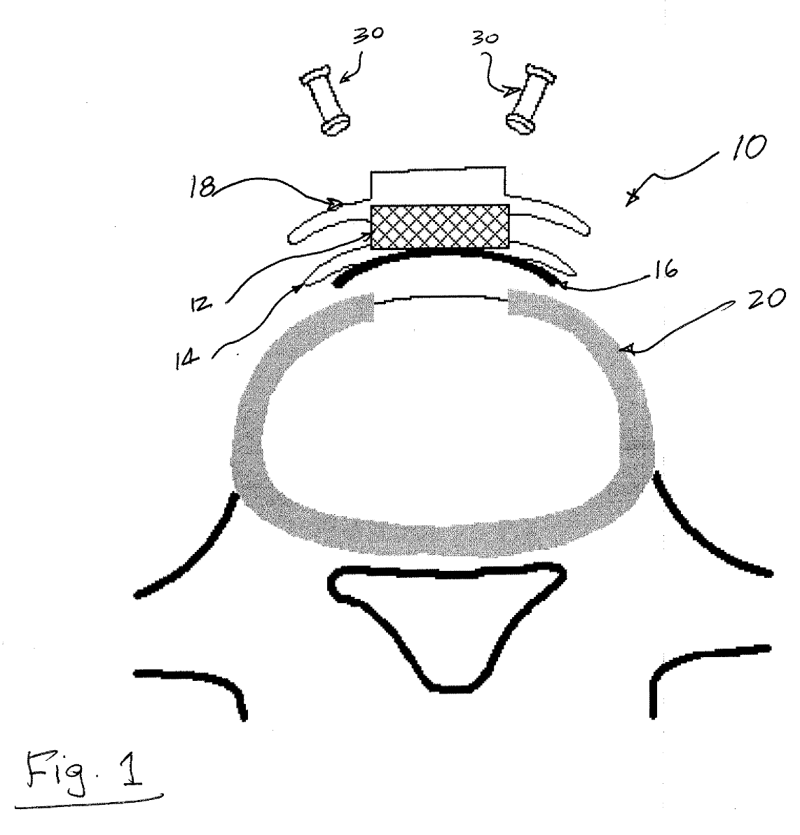

FIG. 1 depicts one embodiment of the device 10 of this invention prior to repair of annulus fibrosus 20. Device 10 comprises a regeneration matrix 12, intra-annular straps 14, intra-annular reinforcement band 16, and extra-annular straps 18. Fasteners 30 are also depicted for securing device 10 to annulus 20.

Regeneration matrix 12 may comprise any biocompatible and bioresorbable material. The bioresorbable material may comprise synthetic and natural materials.

Such materials useful as regeneration matrices 12 in this invention can vary, as long as they provide sufficient strength or characteristics needed to withstand the stresses required to support the intended function of the matrix, cause little or no foreign body reaction and allow the ingrowth of native tissue through the regeneration matrix.

The regeneration matrix can be constructed of the same or different biocompatible materials as readily determined by those of skill in the art. Sufficient strength and physical properties of the regeneration matrix can be developed or achieved through the selection of materials used to form the device and/or from the process used to manufacture the device. In an exemplary embodiment, the device is formed from a bioresorbable or bioabsorbable material, and more preferably from a bioresorbable or bioabsorbable material that has the ability to resorb in a timely fashion in the body environment while providing an initial scaffold for native tissue ingrowth. For example, bioresorbable or bioabsorbable material can preferably resorb in less than a year. For the purposes of this invention, the terms “bioresorbable” and “bioabsorbable” are intended to be used interchangeably and denote a material that that is excreted from the body through normal physiological processes.

Furthermore, one skilled in the art would appreciate that the form that the regeneration matrix can take may vary according to the desired application. For example, the regeneration matrix may take the form of a film, foam, gel, mesh, woven or non-woven matrix.

In one embodiment of the present invention, the regeneration matrix can be formed from a biocompatible polymer. A variety of biocompatible polymers, both bioabsorbable and nonbioabsorbable, can be used as the regeneration matrix according to the present invention. The biocompatible polymers can be synthetic polymers, natural polymers or combinations thereof. As used herein the term “synthetic polymer” refers to polymers that are not found in nature, even if the polymers are made from naturally occurring biomaterials. The term “natural polymer” refers to polymers that are naturally occurring.

In embodiments where the regeneration matrix includes at least one synthetic polymer. Suitable biocompatible synthetic polymers can include polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarate), polyurethane, poly(ester urethane), poly(ether urethane), and blends and copolymers thereof.

Of the foregoing, useful non-bioabsorbable polymers include, but are not limited to polyacrylates, ethylene-vinyl acetates (and other acyl-substituted cellulose acetates), polyester (Dacron®), poly(ethylene terephthalate), polypropylene, polyethylene, polyurethanes, polystyrenes, polyvinyl oxides, polyvinyl fluorides, poly(vinyl imidazoles), chlorosulphonated polyolefins, polyethylene oxides, polyvinyl alcohols (PVA), polytetrafluoroethylenes, nylons, and combinations thereof.

Suitable synthetic polymers for use in the present invention can also include biosynthetic polymers based on sequences found in collagen, laminin, glycosaminoglycans, elastin, thrombin, fibronectin, starches, poly(amino acid), gelatin, alginate, pectin, fibrin, oxidized cellulose, chitin, chitosan, tropoelastin, hyaluronic acid, silk, ribonucleic acids, deoxyribonucleic acids, polypeptides, proteins, polysaccharides, polynucleotides and combinations thereof.

For the purpose of this invention aliphatic polyesters include, but are not limited to, homopolymers and copolymers of lactide (which includes lactic acid, D-,L- and meso lactide); glycolide (including glycolic acid); caprolactone; p-dioxanone (1,4-dioxan-2-one); trimethylene carbonate (1,3-dioxan-2-one); alkyl derivatives of trimethylene carbonate; δ-valerolactone; β-butyrolactone; γ-butyrolactone; ε-decalactone; hydroxybutyrate; hydroxyvalerate; 1,4-dioxepan-2-one (including its dimer 1,5,8,12-tetraoxacyclotetradecane-7,14-dione); 1,5-dioxepan-2-one; 6,6-dimethyl-1,4-dioxan 2-one; 2,5-diketomorpholine; pivalolactone; α,αdiethylpropiolactone; ethylene carbonate; ethylene oxalate; 3-methyl-1,4-dioxane-2,5-dione; 3,3-diethyl-1,4-dioxan-2,5-dione; 6,6-dimethyl-dioxepan-2-one; 6,8-dioxabicycloctane-7-one and polymer blends thereof. Aliphatic polyesters used in the present invention can be homopolymers or copolymers (random, block, segmented, tapered blocks, graft, triblock, etc.) having a linear, branched or star structure. Other useful polymers include polyphosphazenes, co-, ter- and higher order mixed monomer based polymers made from L-lactide, D,L-lactide, lactic acid, glycolide, glycolic acid, para-dioxanone, trimethylene carbonate and ε-caprolactone.

In one embodiment, the regeneration matrix includes at least one natural polymer. Suitable examples of natural polymers include, but are not limited to, fibrin-based materials, collagen-based materials, hyaluronic acid-based materials, glycoprotein-based materials, cellulose-based materials, silks and combinations thereof.

In yet another embodiment, the regeneration matrix includes a naturally occurring extracellular matrix material (“ECM”), such as that found in the stomach, bladder, alimentary, respiratory, urinary, integumentary, genital tracts, or liver basement membrane of animals. Preferably, the ECM is derived from the alimentary tract of mammals, such as cows, sheep, dogs, cats, and most preferably from the intestinal tract of pigs. The ECM is preferably small intestine submucosa (“SIS”), which can include the tunica submucosa, along with basilar portions of the tunica mucosa, particularly the lamina muscularis mucosa and the stratum compactum.

SIS has been described as a natural acellular biomaterial used to repair, support, and stabilize a wide variety of anatomical defects and traumatic injuries. See, for example, Cook® Online News Release provided by Cook Biotech Inc. at “www.cookgroup.com”. The SIS material is derived from porcine small intestinal submucosa that models the qualities of its host when implanted in human soft tissues. Further, it is taught that the SIS material provides a natural scaffold-like matrix with a three-dimensional structure and biochemical composition that attracts host cells and supports tissue remodeling. SIS products, such as OASIS and SURGISIS, are commercially available from Cook Biotech Inc., Bloomington, Ind.

Another SIS product, RESTORE Orthobiologic Implant, is available from DePuy Orthopaedics, Inc. in Warsaw, Ind. The DePuy product is described for use during rotator cuff surgery, and is provided as a resorbable framework that allows the rotator cuff tendon to regenerate. The RESTORE Implant is derived from porcine small intestine submucosa, a naturally occurring ECM (composed of mostly collagen type I (about 90% of dry weight) glycosaminoglycans and other biological molecules), which has been cleaned, disinfected, and sterilized. During seven years of preclinical testing in animals, there were no incidences of infection transmission from the implant to the host, and the RESTORE Implant has not adversely affected the systemic activity of the immune system.

While small intestine submucosa is available, other sources of ECM are known to be effective for tissue remodeling. These sources include, but are not limited to, stomach, bladder, alimentary, respiratory, or genital submucosa, or liver basement membrane. See, e.g., U.S. Pat. Nos. 6,379,710, 6,171,344, 6,099,567, and 5,554,389, hereby incorporated by reference. Further, while SIS is most often porcine derived, it is known that these various submucosa materials may be derived from non-porcine sources, including bovine and ovine sources. Additionally, the ECM material may also include partial layers of laminar muscularis mucosa, muscularis mucosa, lamina propria, stratum compactum and/or other tissue materials depending upon factors such as the source from which the ECM material was derived and the delamination procedure.

For the purposes of this invention, it is within the definition of a naturally occurring ECM to clean and/or comminute the ECM, or to cross-link the collagen within the ECM. It is also within the definition of naturally occurring extracellular matrix to fully or partially remove one or more components or subcomponents of the naturally occurring matrix. However, it is not within the definition of a naturally occurring ECM to extract, separate and purify the natural components or sub-components and reform a matrix material from purified natural components or sub-components. Also, while reference is made to SIS, it is understood that other naturally occurring ECMs (e.g., stomach, bladder, alimentary, respiratory or genital submucosa, and liver basement membrane), whatever the source (e.g., bovine, porcine, ovine) are within the scope of this invention. Thus, in this application, the terms “naturally occurring extracellular matrix” or “naturally occurring ECM” are intended to refer to extracellular matrix material that has been cleaned, disinfected, sterilized, and optionally cross-linked.

The following U.S. patents, hereby incorporated by reference, disclose the use of ECMs for the regeneration and repair of various tissues: U.S. Pat. Nos. 6,379,710; 6,187,039; 6,176,880; 6,126,686; 6,099,567; 6,096,347; 5,997,575; 5,993,844; 5,968,096; 5,955,110; 5,922,028; 5,885,619; 5,788,625; 5,762,966; 5,755,791; 5,753,267; 5,733,337; 5,711,969; 5,645,860; 5,641,518; 5,554,389; 5,516,533; 5,445,833; 5,372,821; 5,352,463; 5,281,422; and 5,275,826.

Another type of ECM is found in U.S. Pat. No. 6,042,610 to ReGen Biologics, hereby incorporated by reference, and discloses the use of a device comprising a bioabsorbable material made at least in part from purified natural fibers. The purified natural fibers are cross-linked to form the device of U.S. Pat. No. 6,042,610. The device can be used to provide augmentation for a damaged meniscus. Related U.S. Pat. Nos. 5,735,903, 5,479,033, 5,306,311, 5,007,934, and 4,880,429 also disclose a meniscal augmentation device for establishing a scaffold adapted for ingrowth of meniscal fibrochondrocytes.

In other embodiments of the present invention, the regeneration matrix can be formed from elastomeric copolymers such as, for example, polymers having an inherent viscosity in the range of about 1.2 dL/g to 4 dL/g, more preferably about 1.2 dL/g to 2 dL/g, and most preferably about 1.4 dL/g to 2 dL/g as determined at 25° C. in a 0.1 gram per deciliter (g/dL) solution of polymer in hexafluoroisopropanol (HFIP). Suitable elastomers also preferably exhibit a high percent elongation and a low modulus, while possessing good tensile strength and good recovery characteristics. In the preferred embodiments of this invention, the elastomer exhibits a percent elongation greater than about 200 percent and preferably greater than about 500 percent. In addition to these elongation and modulus properties, the elastomers should also have a tensile strength greater than about 500 psi, preferably greater than about 1,000 psi, and a tear strength of greater than about 50 lbs/inch, preferably greater than about 80 lbs/inch.

Exemplary biocompatible elastomers include, but are not limited to, elastomeric copolymers of ε-caprolactone and glycolide with a mole ratio of ε-caprolactone to glycolide of from about 35:65 to about 65:35, more preferably from 45:55 to 35:65; elastomeric copolymers of ε-caprolactone and lactide (including L-lactide, D-lactide, blends thereof, and lactic acid polymers and copolymers) where the mole ratio of ε-caprolactone to lactide is from about 95:5 to about 30:70 and more preferably from 45:55 to 30:70 or from about 95:5 to about 85:15; elastomeric copolymers of p-dioxanone (1,4-dioxan-2-one) and lactide (including L-lactide, D-lactide, blends thereof, and lactic acid polymers and copolymers) where the mole ratio of p-dioxanone to lactide is from about 40:60 to about 60:40; elastomeric copolymers of ε-caprolactone and p-dioxanone where the mole ratio of ε-caprolactone to p-dioxanone is from about from 30:70 to about 70:30; elastomeric copolymers of p-dioxanone and trimethylene carbonate where the mole ratio of p-dioxanone to trimethylene carbonate is from about 30:70 to about 70:30; elastomeric copolymers of trimethylene carbonate and glycolide (including polyglycolic acid) where the mole ratio of trimethylene carbonate to glycolide is from about 30:70 to about 70:30; elastomeric copolymers of trimethylene carbonate and lactide (including L-lactide, D-lactide, blends thereof, and lactic acid polymers and copolymers) where the mole ratio of trimethylene carbonate to lactide is from about 30:70 to about 70:30; and blends thereof. Other examples of suitable biocompatible elastomers are described in U.S. Pat. No. 5,468,253.

In another embodiment of the present invention, the regeneration matrix can be formed from an elastomer that is a copolymer of 35:65 ε-caprolactone and glycolide, formed in a dioxane solvent and including a polydioxanone mesh. In another embodiment, the elastomer used to form the tissue repair device can be a copolymer of 40:60 ε-caprolactone and lactide with a polydioxanone mesh. In yet another embodiment, the elastomer is a 50:50 blend of a 35:65 copolymer of ε-caprolactone and glycolide and 40:60 copolymer of ε-caprolactone and lactide. The polydioxanone mesh may be in the form of a one layer thick two-dimensional mesh or a multi-layer thick three-dimensional mesh.

In yet another embodiment of the present invention, the regeneration matrix can be formed from a polymeric foam component having pores with an open cell pore structure.

The pore size can vary, but preferably, the pores are sized to allow tissue ingrowth. More preferably, the pore size is in the range of about 20 to 1000 microns, and even more preferably, in the range of about 20 to 500 microns. The polymeric foam component can, optionally, contain a reinforcing component, such as for example, the textiles disclosed above. In some embodiments where the polymeric foam component contains a reinforcing component, the foam component can be integrated with the reinforcing component such that the pores of the foam component penetrate the mesh of the reinforcing component and interlock with the reinforcing component.

It may also be desirable to use polymer blends to form a regeneration matrix which transitions from one composition to another composition in a gradient-like architecture. For example, by blending an elastomer of ε-caprolactone-co-glycolide with ε-caprolactone-co-lactide (e.g., with a mole ratio of about 5:95) a device may be formed that transitions from a softer spongy material to a stiffer more rigid material. Clearly, one skilled in the art will appreciate that other polymer blends may be used for similar gradient effects, or to provide different gradients (e.g., different absorption profiles, stress response profiles, different degrees of elasticity, or different porosities).

One of ordinary skill in the art will appreciate that the selection of a suitable material for forming the biocompatible tissue regeneration matrix of the present invention depends on several factors. These factors include in vivo mechanical performance; cell response to the material in terms of cell attachment, proliferation, migration and differentiation; biocompatibility; and optionally, bioabsorption (or biodegradation) kinetics. Other relevant factors include the chemical composition, spatial distribution of the constituents, the molecular weight of the polymer, and the degree of crystallinity.

The differences in the absorption time under in vivo conditions can also be the basis for combining two different copolymers when forming the device of the present invention For example, a copolymer of 35:65 ε-caprolactone and glycolide (a relatively fast absorbing polymer) can be blended with 40:60 ε-caprolactone and L-lactide copolymer (a relatively slow absorbing polymer) to form a biocompatible scaffold. Depending upon the processing technique used, the two constituents can be either randomly inter-connected bicontinuous phases, or the constituents could have a gradient-like architecture in the form of a laminate-type composite with a well integrated interface between the two constituent layers.

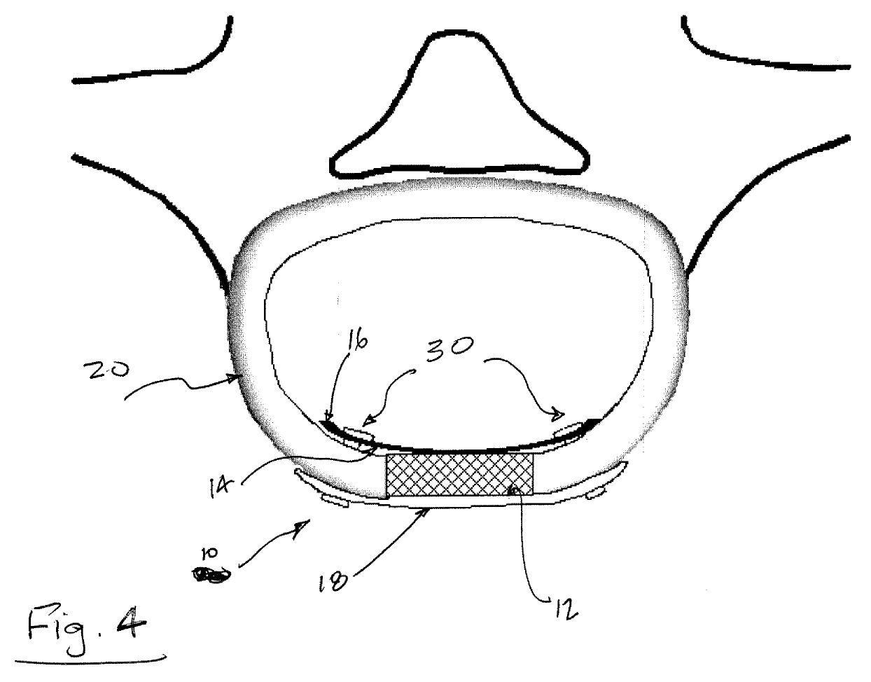

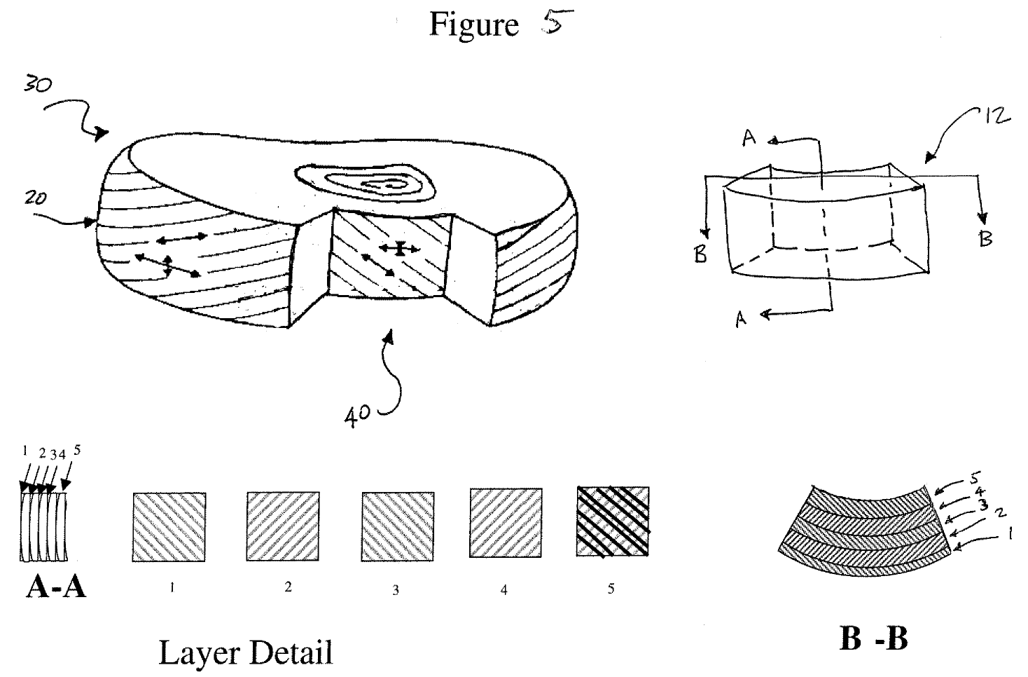

Regeneration matrix 12 preferably is comprised of multiple annular layers. FIG. 4 depicts views of disc 30 and annulus 20 with void 40 into which device 10 is inserted to repair annulus 20. In FIG. 5, generation matrix 12 is further depicted in views A-A and B-B. View A-A is a cross-sectional view of regeneration matrix 12 and is shown to comprise five layers. A further detail shows layers 1 to 5 as made of different materials and orientations. It should be noted that the individual layers of regeneration matrix 12 may be made of the same material, different materials, or of the same material with differing properties such as elasticity or tensile strength. View B-B is a top view of regeneration matrix 12 showing layers 1 to 5.

The annular layers of regeneration matrix 12 may be held together by various means such as by processing means including lamination techniques, pressing, welding, vacuum forming, or by chemical techniques, including is glues, adhesives, surfacants, as well as mechanical techniques including sutures, spikes anchors, fasteners, surface interacting features, roughness, for example.

In a preferred embodiment, regeneration matrix 12 is comprised of laminated layers. For example, extra-cellular matrix (ECM) or fabric based polymers are obtained and sectioned into segments and placed into a holder or compression mold such that the natural orientation of the collagen, fibers, polymer chains, or polymer weave is at a 45 deg angle to the intervertebral disc axial plane (1), the next layer (2) is placed at the opposing angular direction reinforcing the initial layer in a radial fashion. A layer of dispensed glue (e.g., Fibrin Glue, Cyanocrylates, gluteraldhydes, etc) or a contact adhesive strip may be placed between these layers for improved adhesion. A third layer with alternating orientations is placed upon the initial two, followed by a 4^{th} and 5^{th} alternating layer. This process can be continued to incorporate as many layers as desired for appropriate strength and remodeling characteristics. This assembly is subsequently compressed to allow for attachment of the various layers. Heat, vacuum, welding methods (vibrational, ultrasonics, e.g.) can be employed to enhance the matrix intergrity.

Additionally, sutures may be used at varying stages of manufacture to further enhance laminate layer bonding. These sutures can be produced from permanent implant or bioresorbable materials including polypropylene, polyethylene, polyesters, Polydixanone, Polylacide(s), glyoclyde(s), capralactides, for example.

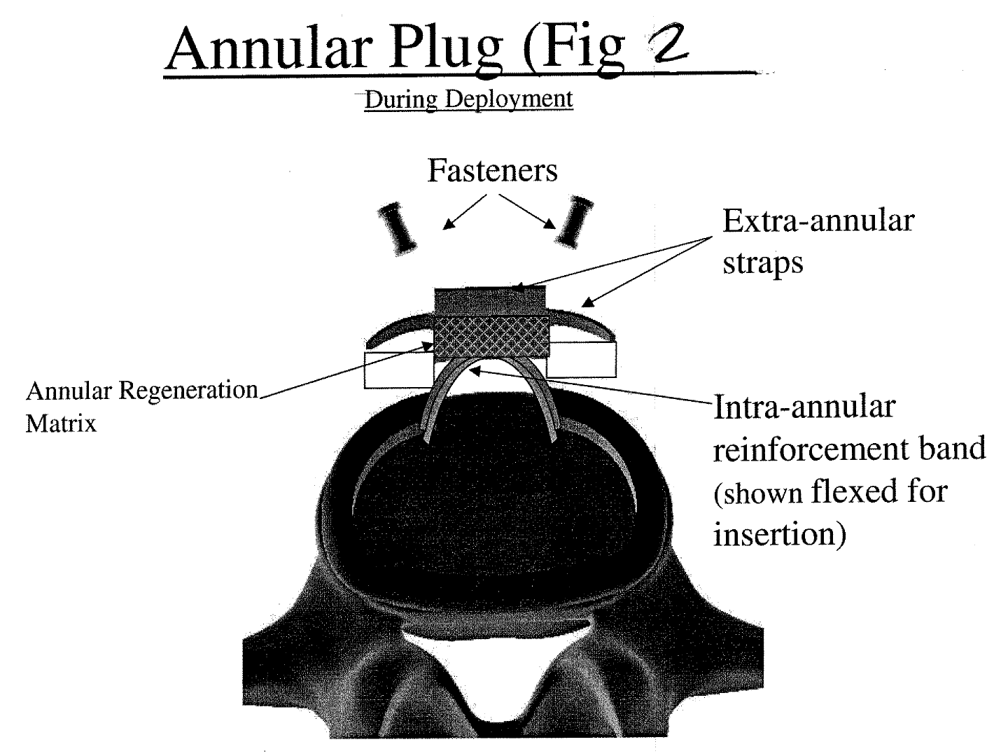

Intra-annular straps 14 and extra-annular straps 18 may be made of the same or different biocompatible materials as described below. It should be noted that intra annular straps 14 and extra-annular straps 18 need not be used in conjunction with each other or otherwise be present in the same device. In other words, device 10 may only require intra-annular straps 14 for certain applications or only require extra-annular straps 18 for other applications. However, the preferred embodiment is to have both intra-annular straps 14 and extra-annular straps 18 used present in the device. It should further be noted that extra-annular straps 18 may comprise straps that project in both the horizontal (18a) and vertical (18b) directions (better shown in FIG. 2).

Suitable examples for materials for intra-annular straps 14 and extra-annular straps 18 may comprise extracelluar matrix from sources and/or allograft, autograft, and xenograft restorable or non-resorbable polymers including polypropylene, polyethylene, polyesters, polydixanone, polylacide(s), glyoclyde(s), capralactides, etc.

Reinforcement band 16 is an optional segment of device 10 and functions to apply a force to intra-annular strap 14 to firmly press strap 14 against the inner wall of annulus fibrosus 20. Preferably, reinforcement band 16 is biased to extend as substantially depicted in FIGS. 1 and 2, i.e., substantially coextensive with intra-annular strap 14.

Suitable material for reinforcement band 16 include a material that offers short term memory capabilities including memory polymers (polypropylene, polyethylene, for example or metallics (Nitinol, stainless steel sheets, Ti6Al4V, for example).

One material exhibiting shape memory or super-elastic characteristics is Nitinol.

Nitinol is utilized in a wide variety of applications, including medical device applications as described above.

Nitinol or NiTi alloys are widely utilized in the fabrication or construction of medical devices for a number of reasons, including its biomechanical compatibility, its biocompatibility, its fatigue resistance, its kink resistance, its uniform plastic deformation, its magnetic resonance imaging compatibility, its ability to exert constant and gentle outward pressure, its dynamic interference, its thermal deployment capability, its elastic deployment capability, its hysteresis characteristics, and is moderately radiopaque.

Nitinol, as described above, exhibits shape memory and/or super-elastic characteristics. Shape memory characteristics may be simplistically described as follows. A metallic structure, for example, a Nitinol tube that is in an Austenitic phase may be cooled to a temperature such that it is in the Martensitic phase. Once in the Martensitic phase, the Nitinol tube may be deformed into a particular configuration or shape by the application of stress. As long as the Nitinol tube is maintained in the Martensitic phase, the Nitinol tube will remain in its deformed shape. If the Nitinol tube is heated to a temperature sufficient to cause the Nitinol tube to reach the Austenitic phase, the Nitinol tube will return to its original or programmed shape. The original shape is programmed to be a particular shape by well-known techniques.

Super-elastic characteristics may be simplistically described as follows. A metallic structure for example, a Nitinol tube that is in an Austenitic phase may be deformed to a particular shape or configuration by the application of mechanical energy. The application of mechanical energy causes a stress induced Martensitic phase transformation. In other words, the mechanical energy causes the Nitinol tube to transform from the Austenitic phase to the Martensitic phase. By utilizing the appropriate measuring instruments, one can determined that the stress from the mechanical energy causes a temperature drop in the Nitinol tube. Once the mechanical energy or stress is released, the Nitinol tube undergoes another mechanical phase transformation back to the Austenitic phase and thus its original or programmed shape. As described above, the original shape is programmed by well know techniques. The Martensitic and Austenitic phases are common phases in many metals.

Medical devices constructed from Nitinol are typically utilized in both the Martensitic phase and/or the Austenitic phase. The Martensitic phase is the low temperature phase. A material is in the Martensitic phase is typically very soft and malleable. These properties make it easier to shape or configure the Nitinol into complicated or complex structures. The Austenitic phase is the high temperature phase. A material in the Austenitic phase is generally much stronger than the material in the Martensitic phase. Typically, many medical devices are cooled to the Martensitic phase for manipulation and loading into delivery systems. When the device is deployed at body temperature, they return to the Austenitic phase.

Other materials that have shape memory characteristics may also be used, for example, some polymers and metallic composition materials. It should be understood that these materials are not meant to limit the scope of the invention.

FIG. 2 depict device 10 during deployment or insertion to repair annulus fibrosus 20. Specifically intra-annular strap 14 and reinforcement band 16 are flexed to allow passage of strap 14 and band 16 through the defect in annulus fibrosus 20.

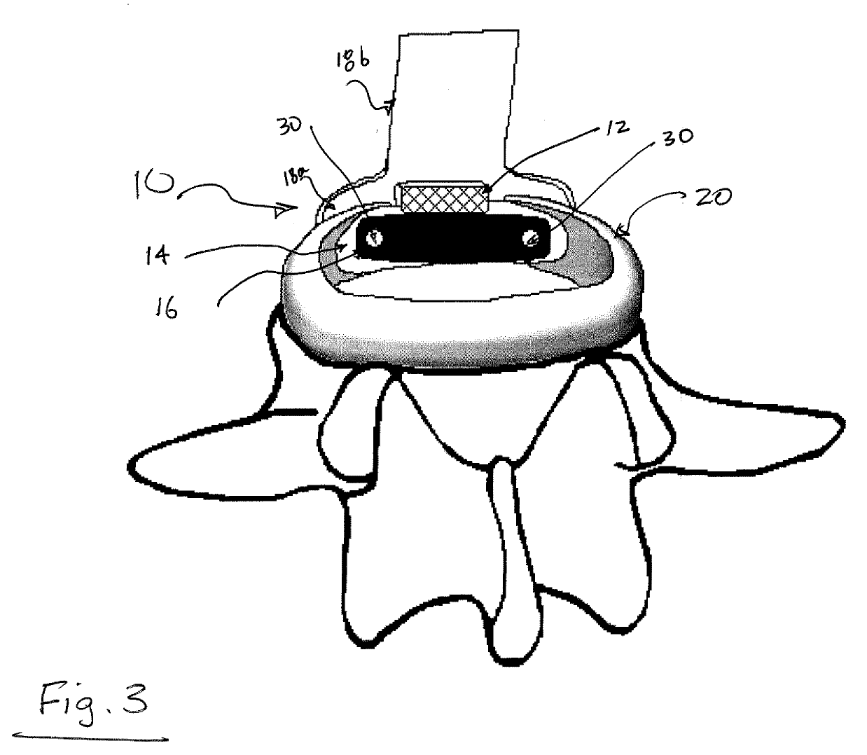

FIG. 3 depicts a view of device 10 deployed to repair and regenerate annulus fibrosus 20. Extra-annular straps 18 are shown to comprise horizontal extra-annular straps 18a and vertical extra-annular straps 18b. Fasteners 30 are shown securing device 10 to annulus fibrosus 20.

FIG. 4 is a top down view of FIG. 3, wherein fasteners 30 penetrate extra-annular strap 18, annulus fibrosus 20, intra-annular strap 14, and reinforcement member 16.

In use, the intra-annular straps are placed within the disc space, the annular wall regeneration matrix is seated between the vertebral body endplates and the intra-annular and extra-annular straps. The extra-annular straps are affixed to the superior and inferior vertebral bodies.

Preferably the regeneration matrix and the straps are comprised of collagen, synthetic or organic polymers or other biologically regeneratable material. The preferred embodiment employs an Extracellular Matrix (ECM) formed from processed SIS with radial layer, slurry or foam construction that has been enhanced with growth factors, stem cells, and/or GAGs to facilitate annular regeneration. The matrix may also utilize hydrogels or other hydroscopic material to regain/maintain disc height or utilize biological lubricants (hylauronic acid, for example) to facilitate lubrication of tissue and/or intervertebral devices.

The fasteners (rivets, screws, anchors, staples, for example) and bands are produced from of synthetic or organic polymers or other biologically regeneratable material. The preferred embodiment of the band employs resorbable polylatic/glycolic copolymer with properties to allow for flexion upon insertion into the disc space and expansion to the inner annular wall upon release (see FIGS. 2 and 3). The bands can be fabricated within the intra-annular straps and may include through holes to receive fasteners.

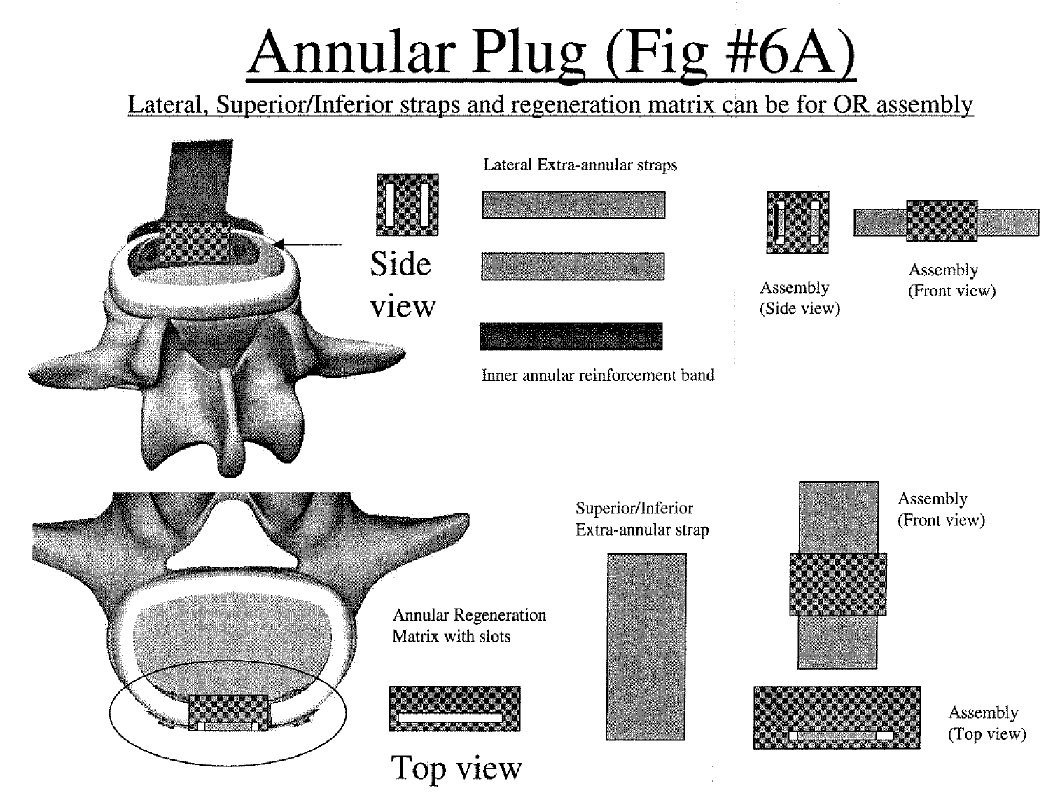

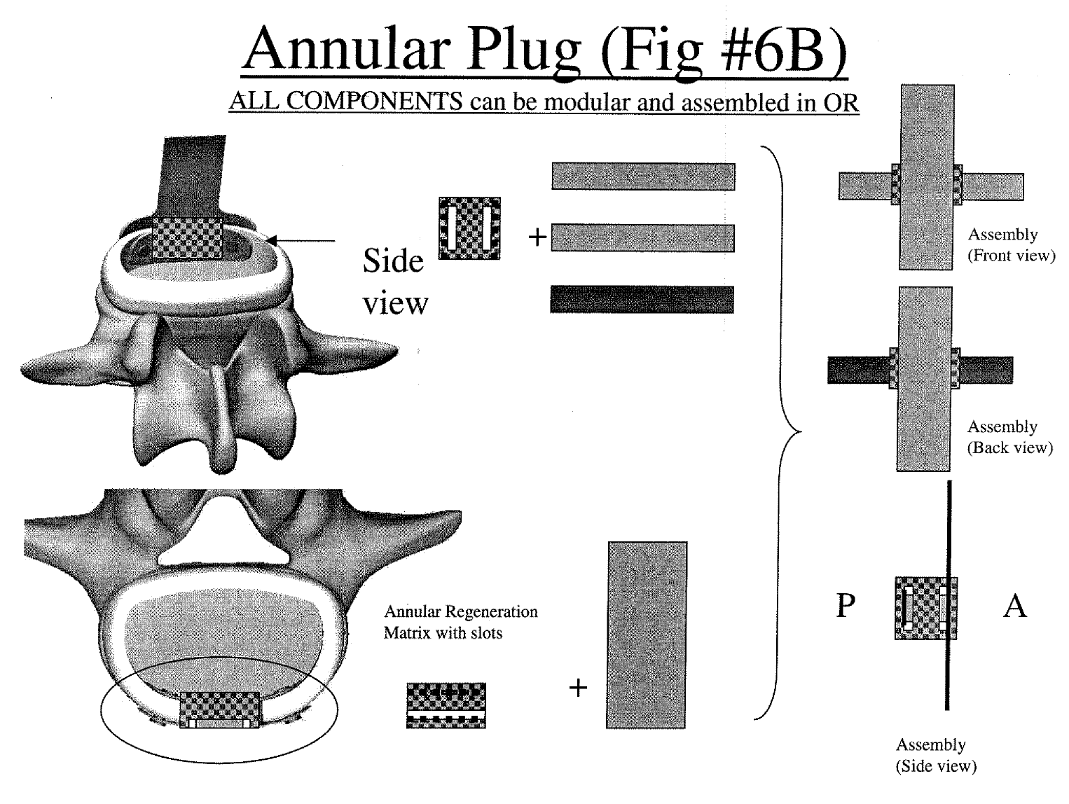

In another embodiment device 10 of this invention may be modular wherein the annular straps and reinforcement bands are passed through regeneration matrix to allow for multiple size and configurations based upon location, procedure, and defect size as shown in FIGS. 6a and 6b.

Referring to FIG. 6a, device 10 is depicted in unassembled, assembled, and deployed views and comprises the modular components of regeneration matrix 12 having slits 11 for passing lateral intra-annular straps 14 and/or reinforcement band 16 and for passing lateral extra-annular straps 18a and/or superior/inferior extra-annular straps 18b.

FIG. 6b provides yet another set of views for the unassembled, assembled, and deployed views.

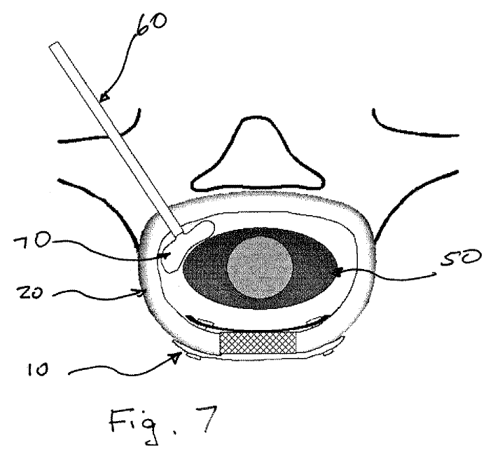

The device 10 of this invention is also suited for use in methods to repair the nucleus pulposus or in revision surgeries involving artificial discs. In particular in the case of a defective artificial disc and as show in FIG. 7, a defective annulus 20 may be repaired by device 10 and a minimally invasive cannula or trochar 60 may be used to inject stabilizing material 70 to immobilize the defective implant 50.

Thus an important advantage of the device of this invention is a method to revise failed nucleus or disc replacement which is facilitated following use of the annulus fibrosus repair device as the annulus is sealed and regenerated. Stabilizing material 70 may comprise osteoconductive and/or osteoinductive materials including calcium- or phosphate-based or derived cements, mineralized collagen, auto/allo/xenograft pastes, stem cells, bone morphogenic proteins (BMP's), bone marrow, bioactive glasses and growth differentiation factors, for example for annular calcification and fusion along with optional posterior screws/instrumentation.

Yet another embodiment of this invention is a kit comprising the sterile components of various sizes useful for assembling the device of the invention which will allow for the convenient assembly of the invention in a surgical setting with modular components. In a preferred embodiment, a kit of the invention will provide sterile components suitable for easy use in the surgical environment, and will provide slotted regeneration matrices slotted for receipt of further sterile kit components of intra-annular straps, extra-annular straps, reinforcement bands and fasteners. In a preferred embodiment, a kit of the invention contains various sizes of an slotted SIS regeneration matrix (slotted for receipt of intra- and extra-annular straps and reinforcement bands), with various sizes of extra-annular and intra-annular straps comprised of SIS, and various sizes of a reinforcement band comprised of nitinol, and fasteners comprised of SIS. As would be apparent to one of skill in the art, the foregoing components may be comprised of any of the materials hereinbefore disclosed as suitable for use as the appropriate component.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A device for repair and/or regeneration of the annulus fibrosus of an intervertebral disc comprising:
a) a regeneration matrix sized and shaped to form a part of the annulus fibrosus;
b) the regeneration matrix further comprising a strap or projection to secure the material to the annulus fibrosus.

2. The device of claim 1, wherein the strap comprises an intra-annular strap.

3. The device of claim 1, wherein the strap further comprises an extra-annular strap.

4. The device of claim 2, wherein the intra-annular strap further comprises a reinforcement band.

5. The device of claim 4, wherein the device further comprises fasteners for fastening the straps and reinforcement band to the annulus fibrosus.

6. The device of claim 5, further comprising extra-annular straps in the longitudinal direction along the length of the spine and wherein the longitudinal extra-annular straps are fastened by fasteners to adjacent intervertebral bodies.

7. The device of claim 1 wherein the regeneration matrix comprises extracellular matrix.

8. The device of claim 7, wherein the extracellular matrix is small intestine submucosa.

9. A kit comprising slotted regeneration matrices slotted for receipt of further sterile kit components of intra-annular straps, extra-annular straps, reinforcement bands and fasteners.

10. The kit of claim 9, wherein the regeneration matrices, intra- and extra-annular straps, and fasteners are comprised of SIS and the reinforcement bands are comprised of nitinol.

11. A method of stabilizing a defective intervertebral nucleus implant in an intervertebral disc with a defective or damaged annulus fibrosus comprising the steps of:
a) sealing the defective portion of the annulus fibrosus of an intervertebral disc with an annulus fibrosus repair device; and
b) injecting a stabilizing material into the intervertebral disc space.

12. The method of claim 11, wherein step b is accomplishing by inserting a trochar or cannula through a portion of the annulus fibrosus.

13. The method of claim 11, wherein step b is accomplished by injecting through the annulus fibrosus repair device.

14. The method of claim 11, wherein the stabilizing material is selected from a osteoconductive or osteoinductive material.

15. The method of claim 14, wherein the stabilizing material is selected from the group consisting of calcium- or phosphate-based or derived cements, mineralized collagen, auto/allo/xenograft pastes, stem cells, bone morphogenic proteins (BMP's), bone marrow, bioactive glasses and growth differentiation factors.
